(19) United States
(12) Patent Application Publication
(10) Pub. No.: US 2002/0172729 A1
(43) Pub. Date: Nov. 21, 2002

(54) Pharmaceutical composition for stabilising atherosclerotic plaques

(75) Inventors: Kalevi John Kenton, London (GB); Adam Henry Carey, London (GB); Beverly Jane Carey, London (GB); Antony John Haynes, London (GB)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Avansis Limited (formerly Kenton Corporation LTD), London (GB)

(21) Appl. No.: 10/118,964

(22) Filed: Apr. 10, 2002

Related U.S. Application Data
(62) Division of application No. 09/669,597, filed on Sep. 26, 2000.

(30) Foreign Application Priority Data
Sep. 27, 1999 (GB) 99 22751.4

(51) Int. Cl. A61K 35/78; A61K 31/7048; A61K 31/355; A61K 31/375; A61K 31/353; A61K 31/198; A61K 31/015

(52) U.S. Cl. 424/757; 514/27; 514/456; 514/458; 514/474; 514/423; 514/562; 514/564; 514/565; 514/763

(57) ABSTRACT

The invention relates to a pharmaceutical composition that can be used to treat or prevent disorders of the vascular system. The composition comprises lycopene in combination with a flavonoid, an amino acid, magnesium, ascorbate and vitamin E.

The present invention relates to a pharmaceutical composition that can be used to treat or prevent disorders of the vascular system resulting from the presence in the blood vessels of thrombi and emboli that may arise from the dissociation of atherosclerotic plaques. In particular, it relates to the use of lycopene in combination with a flavonoid, an amino acid, magnesium, ascorbate and vitamin E for the preparation of a medicament for therapeutic application in humans and animals.

Plaque formation occurs mainly but not exclusively in the arterial system and arterial atherosclerosis is a major factor in various forms of occlusive vascular diseases including angina, coronary insufficiency, myocardial infarction, heart failure, arterial hypertension, arterial aneurysms, impaired kidney and liver and retinal function, intermittent claudication, cerebral insufficiency, and stroke. Unstable plaques can ulcerate, rupture and the emboli formed can occlude blood vessels causing thromboembolic disease such as coronary thrombosis, stroke and pulmonary embolism. Taken together these diseases are the major cause of premature death and morbidity in the developed world.

Plaques are complex structures that result initially from deposits on the vascular wall that become organised and actively invade the structures of the blood vessels altering their properties and impairing their function. The process of plaque formation follows a well-defined course. It is initiated by an insult (damage) to the endothelium, the layer of cells lining the blood vessel, followed by an inflammatory response that is a precursor of the repair process. The damage may be initiated by turbulence in the blood flow; by adherence of lipoprotein particles to the endothelium particularly if they are oxidised by mechanical trauma, free radical attack or combinations of these. This damage releases small molecular weight mediators, cytokines, and other larger molecules, e.g. C-reactive protein, these all act as chemo-attractants that attract formed elements (cells) from the circulating blood in an effort to repair the damage. The cells that are attracted are initially blood platelets and white blood cells. Circulating dissolved fibrinogen is converted to fibrin by clotting factors, such as thrombin, also released at the damage site. The complex of fibrin and cells can trap further circulating lipid particles that result in the formation of atherosclerotic plaques. Smooth muscle cells from the vessel wall can migrate into the plaque that can then extend into the lumen of the vessel, calcification may then occur leading to the formation of an ultra sound-opaque plaque. Ulceration and bleeding into a plaque may lead to its rupture, which can result in the formation of fragments, emboli, that can be carried into vessels of smaller diameter leading to vascular occlusion, tissue anoxia and tissue death, infarction. The plaque can also serve as a focus for platelet aggregation and leucocyte accretion, the resulting thrombus can break free and travel to a narrow or stenosed vessel where it may occlude the vessel leading to local infarction. If this occurs in the coronary artery the resulting myocardial infarction can produce a heart attack that may be fatal; a similar occurrence in the brain can produce cerebral infarction resulting in a stroke that may prove fatal.

There is a substantial body of evidence to show that the process of atherosclerotic plaque formation often begins at an early age and may be found in children as young as five years old. This may be due to genetic factors or to an unsuitable diet or a combination of both. Plaque formation has been linked with dietary patterns and other environmental factors e.g. smoking. Excess of dietary saturated fats, deficiencies in antioxidant vitamins and lack of exercise have all been implicated in cardiovascular disorders characterised by an increase in plaque formation. By the time atherosclerosis is diagnosed considerable damage to the blood vessel wall has occurred and there is an increased risk of plaque dissociation, thrombus formation and the production of emboli. These can be transported to distant sites and by lodging in the vessel lumen occlude it and lead to local tissue anoxia and cell death (infarction).

Once plaques have formed and become consolidated they remain and are resistant to removal by drug treatment. Surgical removal is possible but hazardous and is only suitable for certain vessels. Angioplasty can stretch the vessel wall and increase the size of the lumen and improve blood flow through the vessel but this procedure carries significant risk and is unlikely to produce a permanent improvement.

Plaque size and density can be measured in blood vessels obtained at post-mortem and this method has been used to demonstrate early plaque formation in children as young as five. Plaques develop progressively with age and with advancing age they become larger and lead to the diseases mentioned above. Modem imaging techniques and MRI scan can now be used in living subjects to determine plaque size and stability accurately. Ultrasound imaging has been shown to be a reliable and cost-effective method for measuring the stability, density, growth, and regression of atherosclerotic plaques. It can be used to measure the efficacy of treatments directed towards stabilising and/or reducing plaque size and density.

Percutaneous transluminal angioplasty (PTA) is a surgical technique used to enlarge the lumen of blood vessels that have been reduced by plaque formation, (stenosis). Following PTA, restenosis may develop as a result of new plaque formation leading to a reduced blood flow with consequent functional impairment of the tissues and organs distal to the stenosis. The formulation described in this invention may be used to prevent restenosis of blood vessels following PTA or other surgical intervention.

Various multivitamin formulations are widely available to be used by those who are likely to lack vitamins, e.g. pregnant women and the elderly. Various formulations containing vitamins and amino acids are also known. EP-625 312 B describes compositions containing lycopene, an amino acid, ascorbic acid, vitamin E, carbohydrates and an organic acid, as a nutrient formulation particularly suitable for those taking part in sports. However this formulation is to be administered in relatively low dosages and contains β-carotene which has been linked to an increased incidence of cancer. U.S. Pat. No. 5,278,189 describes a method for the prevention and treatment of occlusive cardiovascular disease by the administration of ascorbate, a variety of synthetic and natural lipoprotein (a) binding inhibitors and antioxidants. The treatment is said to inhibit the binding of lipoprotein (a) to blood vessel walls and is aimed particularly at atherosclerosis and thrombosis.

It has now been found that a pharmaceutical composition comprising lycopene, a flavonoid, an amino acid, magnesium, ascorbate and vitamin E when administered daily to a human can stabilize atherosclerotic plaques, i.e. prevent their breakdown, inhibit their growth thus permitting the natural repair process to reduce the size of the plaques and may even prevent plaque development.

According to the invention in one aspect there is provided a pharmaceutical composition for stabilizing atherosclerotic plaques, preventing new plaques forming and resolving existing atherosclerotic plaques comprising lycopene or related carotenoid other than β-carotene, at least one flavonoid, an amino acid or derivative or precursor thereof, magnesium, ascorbate and tocopherol (vitamin E) or a derivative, precursor or isomer thereof.

Such a composition is usually presented in unit dosage form.

The composition may be used for the manufacture of a medicament for the treatment of thromboembolic disease, in particular for the stabilization and prevention of atherosclerotic plaques.

According to another aspect of the invention there is provided a method of treating thromboembolic disease, in particular stabilizing atherosclerotic plaques, reducing the size of such plaques and preventing the formation of new plaques.

The lycopene used in the composition is preferably L-lycopene or a related carotenoid e.g. lutein, zeaxanthin. Lycopene, unlike β-carotene, is not transformed into vitamin A in the body.

The composition preferably contains a mixture of flavonoids, e.g. rutin, hesperedin, iproflavone or a bioflavonoid mixture, flavones, flavonals, isoflavones or a mixture thereof.

The amino acid may be selected from lysine, arginine, proline or methionine or a suitable derivative or precursor of any one thereof, such as an ester or salt. Alternatively, a mixture of any two or more of the foregoing may be used. Suitable derivatives or precursors of the amino acid include salts, esters or complexes thereof, e.g. salts with an organic or inorganic acid, for example hydrochloric acid. The preferred amino acid is lysine, particularly L-lysine, which may be present as its monohydrochloride salt or as a magnesium salt or chelate.

The magnesium is preferably provided in the form of a magnesium salt, and conveniently may be the magnesium salt of ascorbic acid.

The ascorbate may be provided in the form of ascorbic acid or a suitable derivative or precursor thereof including salts such as metallic salts, preferably alkaline earth metal salts. Other suitable derivatives or precursors include esters, e.g. that ester known as Ester C(RTM) (Ester C is a formulation of calcium ascorbate and theronic acid, a metabolite of ascorbic acid, and is claimed to produce high and long lasting blood levels of vitamin C). Preferably the ascorbate is in the form of Ester C or of a salt, especially the magnesium salt. The use of a salt renders the ascorbate less irritant to the stomach than the free acid, particularly when used in relatively large quantities.

Suitable derivatives, precursors or isomers of vitamin E include esters thereof, e.g. the acetate, tocotrienols and other forms of tocopherol, e.g. d,l α-tocopherol and preferably d-α-tocopherol, which is also known as R,R,R-alpha-tocopherol.

The composition may include additional components such as garlic, e.g. in the form of garlic powder, coenzyme Q10 and other nutrients such as lipoic acid.

It is to be understood that all of the components used in the invention are to be bio-available when in the body, and to be physiologically acceptable.

In a preferred form the pharmaceutical composition of the invention comprises a mixture of L-lysine or salt, ester or complex thereof; L-magnesium ascorbate or L-ascorbic acid or a salt or ester thereof (vitamin C); α-tocopherol or an isomer or derivative thereof (vitamin E); lycopene (ψψ carotene) or related carotenoid other than β-carotene, coenzyme Q10 and bioflavonoid mix or complex together with a pharmaceutically acceptable carrier thereof. Such a combination of constituents may act synergistically.

The pharmaceutical formulation may be in the form of compressed tablets, filled capsules of gelatine or a vegetable equivalent e.g. modified cellulose or agar, a powder which may be contained in sachets or in the form of a liquid suspension or solution.

Alternatively the pharmaceutical composition may be mixed with other nutritional or non-nutritional carriers and be incorporated into a nutritional product such as a fruit or a fibre bar or wafer.

The formulation may be combined with other pharmaceutical excipients and/or surface coatings to provide sustained release of the active ingredients.

The pharmaceutical composition may comprise from 20 to 60% by weight of amino acid or a salt or ester thereof, from 20 to 60% ascorbic acid or salt or ester thereof and/or 20 to 60% magnesium ascorbate; from 5 to 20% of tocopherol isomers or ester thereof, from 0.2 to 1% of lycopene or ester thereof and from 4 to 12% of flavonoids or complex thereof, and if magnesium ascorbate is not present, 2 to 6% of a magnesium compound.

These percentages are w/w and are expressed as the free component and will, of course, vary should derivatives or precursors be used. Any balance can be made up to 100% by other inert pharmaceutically acceptable materials.

The preferred daily dose of the various components of the composition is as follows:

L-lycopene 3 to 16mg, preferably about 10 mg

L-ascorbic acid 3,000 to 9000, mg, preferably about 6,000 mg

Magnesium 300 to 900 mg, preferably about 600mg

L-lysine as the monohydrochloride 3,000 to 9000, mg, preferably about 6,000 mg

Vitamin E as dl-α-tocopheryl acetate 300 to 900 mg, preferably about 600 mg

At least one flavonoid or a mixture thereof, 600 to 1,800 mg, preferably about 1,200 mg.

The daily dose may be administered as divided doses, i.e. 2, 3 or 4, times a day.

The components of the invention may be used in admixture with one or more pharmaceutically acceptable adjuvants, diluents or carriers. They may be made up into unit doses, e.g. as tablets, capsules or sachets. The preferred daily and unit doses are rather large, and indeed are much larger than conventional doses of vitamins which have been used as nutritional supplements, and these larger doses form another aspect of our invention. Conveniently the components of the invention may be made up as a powder which can be combined with foods, e.g. sprinkled over the foods, or incorporated into, e.g. a fruit or fibre bar or wafer. Alternatively they may be dissolved or suspended in water to form a drink or may be made up in controlled or sustained release form. The components may, if desired, be combined with a suitable flavouring, with trace elements and/or dietary supplements.

The mixtures and compositions according to the invention may be made in the conventional manner known per se.

According to a still further feature of the invention there is provided a pharmaceutical composition in unit dosage form such as a sachet comprising from 150 to 450 mg dl-α tocopheryl acetate preferably from 200 mg to 400 mg, most preferably 300 mg; from 1.5 to 8.0 mg lycopene, preferably 2.0 to 6.0 mg, most preferably 5 mg; from 1500 to 4500 mg L-lysine, preferably 2000 to 4000 mg, most preferably 3000 mg; from 1600 to 5000 mg magnesium ascorbate, preferably 2000 to 4000 mg, most preferably 3300 mg; from 300 to 900 mg bio-flavonoid mixture, preferably 400 to 800 mg, most preferably 600 mg. In general we prefer to use a higher dose when the patient is suffering from a more severe condition. However the higher doses may be used for all types of patients.

Preferably two sachets are taken daily. If, however, the pharmaceutical composition is formulated as a tablet or capsule then each tablet or capsule would comprise about 10% of the unit dosage given above so as to make it of a size acceptable to the patient or consumer.

The pharmaceutical formulation may be co-administered with other drugs to further enhance the beneficial effects of the invention. These may include agents designed to lower blood lipids, such HMCoG reductase, inhibitors collectively known as statins, fibrates and nicotinic acid and its derivatives or drugs acting on platelet function, e.g. aspirin and clopidogrel, and non-steroidal anti-inflammatory agents, e.g. ibuprofen, that may modify the inflammatory process induced by the release of inflammatory mediators.

The continued administration of the composition according to this invention over time could lead to the resolution of plaques and partial or complete restoration of vascular function and reversal of the disease processes listed below.

Thus, the composition according to this invention can be used to prevent and treat a range of diseases that develop following plaque formation and are associated with plaque instability, for example angina, coronary insufficiency, myocardial infarction, arterial hypertension, arterial aneurysms, intermittent claudication, cerebral insufficiency, stroke and retinal thrombosis.

The invention is illustrated, but in no way limited by the following Examples:

### EXAMPLE 1

Method

The following sachet formulation was administered twice a day:

Magnesium ascorbate 3 g

Vitamin E (emulsified) 300 iu (300 mg)

Lysine 3 g

Lycopene 5 mg

Bioflavonoids 600 mg

The following control was used:

As an inactive placebo

One sachet of inert ingredients matching the test material.

The components were emulsified and freeze dried, mixed with orange extract and packaged in a foil sachet. The resulting powders were mixed with water to give an orange flavoured drink.

A controlled double blind clinical trial was set up with two arms, one using the above active composition, one using placebo as set out above. A total of 130 healthy male subjects was recruited, with 65 subjects in each arm. Each subject had one or more small to medium sized plaques (types I to III) in the carotid or femoral artery. The chosen subjects were aged 45 to 65, not taking any medication (including high dose vitamins) and had a cholesterol level of <6.5 mmol/L.

Each subject was asked to take the relevant sachet twice a day.

The ultrasonic plaque character of the relevant artery was measured at the beginning of the trial, and again after one year using the methods developed by Nicolaides (J. Vasc. Surg., 29, 110-9, 1999; Eur. J. Endovasc. Surg, 16, 223-30, 1998).

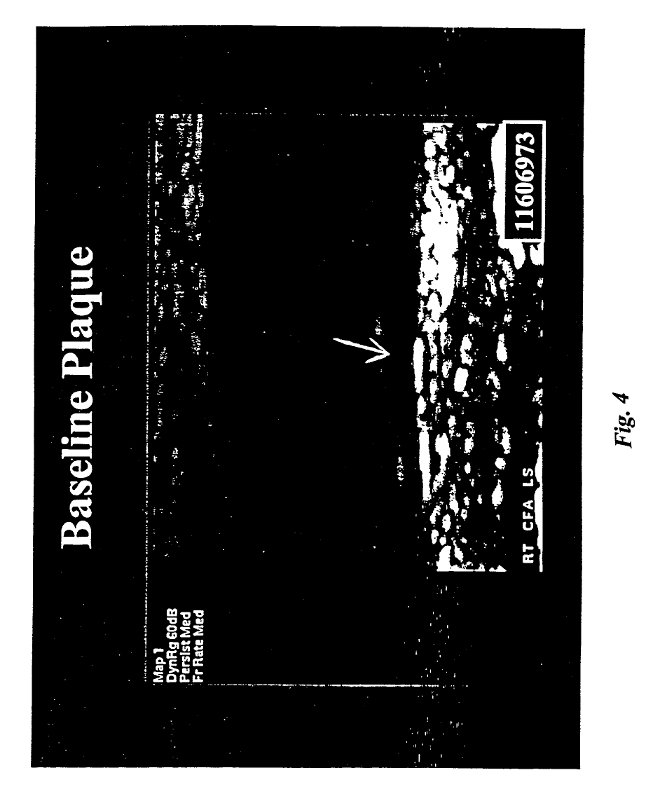

Plaque formation in the carotid and femoral arteries was detected and analysed using an ultra sound scanner imaging apparatus manufactured by ATL Ultrasound USA model HDI 3000 systems. one year of the trial. The white arrow is pointing to the same plaque as shown in FIG. 4, however, the plaque has become more dense showing that it has become more stable.

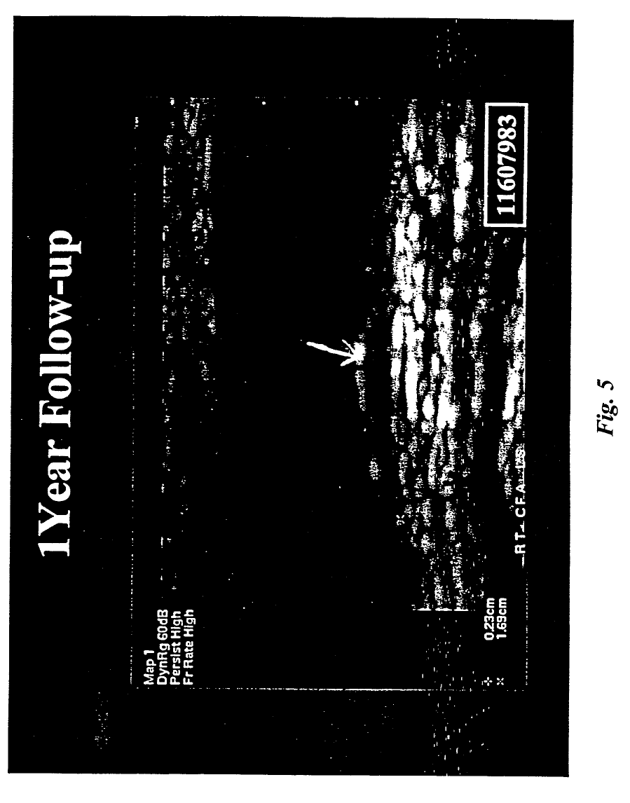

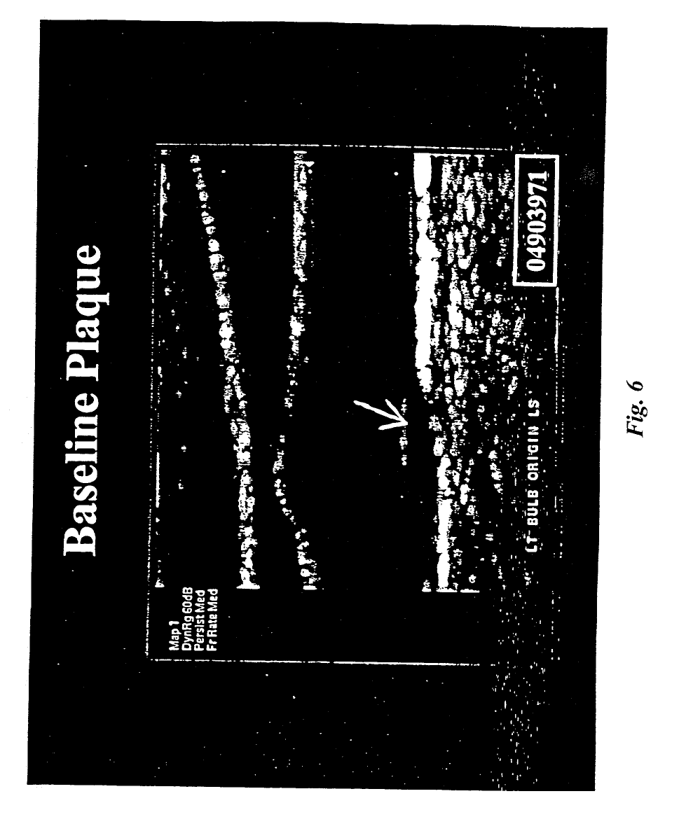

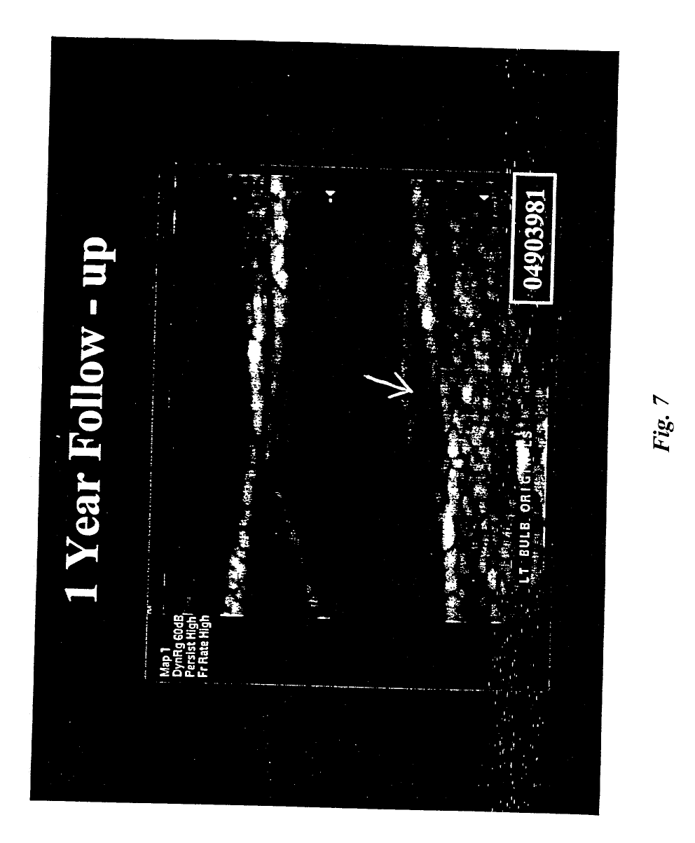

FIGS. 6 and 7 are ultrasonagrams, similar to FIGS. 4 and 5, but showing the effect on a plaque in the left carotid artery of a subject after twelve months of the trial.

Conclusion

Most cardiac events and strokes occur when a softer portion of plaque, ie echolucent, breaks loose and causes an embolism, thereby blocking the blood flow in vessels further down the line.

The results after twelve months of treatment demonstrate that plaque density has been stabilized, thus reducing the risk of a heart attack or a stroke.

### EXAMPLE 2

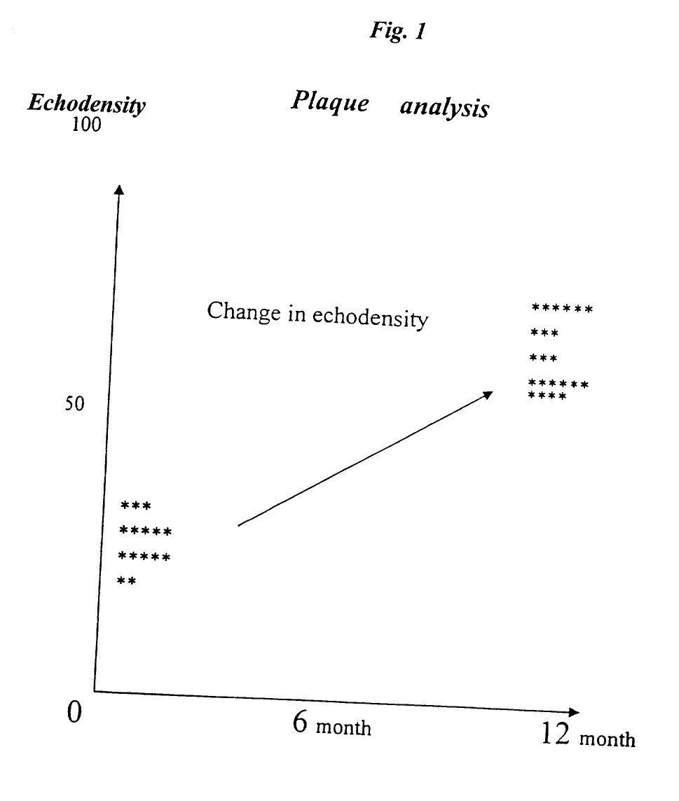

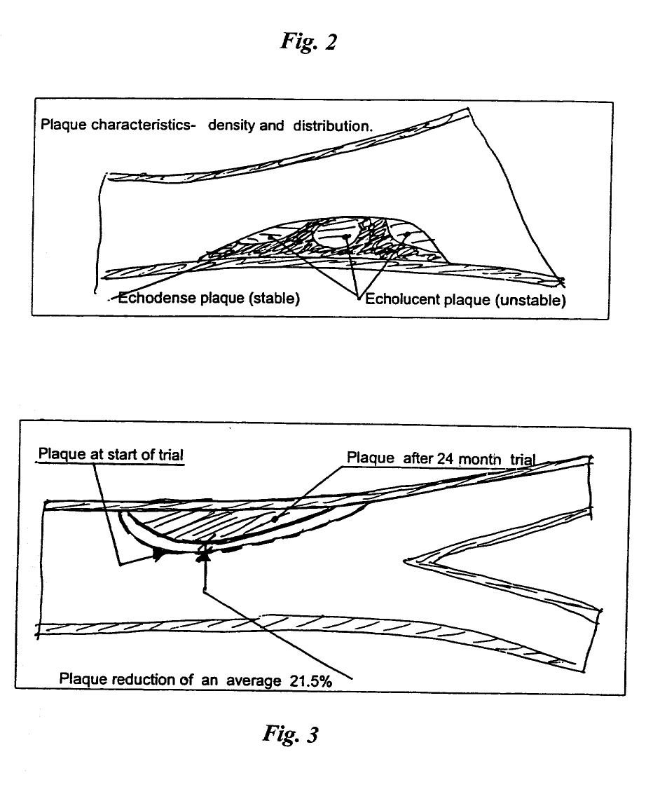

In a similar but smaller study, the plaque sizes were analysed over a period of 24 months. Eleven patients showing evidence of atheroma were administered the active composition described in Example 1 and using the same regime as in Example 1. Using the ultrasound procedure described in Example 1, it was discovered that the average height of plaques was reduced by an average of 21.5% in the subjects who were put on the active compositions versus no reduction in the placebo arm. When calculating the total volume of the plaque the reduction was estimated to a reduction of about 50% (see FIG. 3).

### EXAMPLE 3

Subjects and Methods

Thirty healthy male volunteers were recruited from respondents to articles in the national press.

Inclusion criteria

Male

Age 45 to 63 years inclusive

Fasting serum cholesterol >6.5 mmol/1 but below the Standing Medical Advisory

Committee's recommendations for the use of statins (Calman, K 1997, Dept of

Health CMO's Update 15, p3).

Exclusion criteria

Smokers

Previous history of:

cardiovascular disease

diabetes mellitus

thyroid disease

Hypertension

Concomitant use of any of the following:

mega-dose antioxidant supplementation

(eg >400 iu vitamin E or >1000 mg vitamin C)

antihypertensives

low dose aspirin

lipid lowering agents

Investigations

Potential subjects were screened by taking a medical history and fasting serum lipid profile. Those who fulfilled the inclusion criteria were invited to take part in the study. Measurements were taken of height, weight and blood pressure.

Subjects were randomly assigned to receive either the active composition or the placebo as in Example 1. They were reviewed every four weeks for three months when the above investigations were repeated.

At the follow up visits subjects were asked to return any unused “medication” as a check of compliance and were questioned about adverse events.

Statistical Analysis

The levels of the measured lipid variables at entrance to the study in the active group were compared to the corresponding levels at entrance in the placebo group using a students t-test. The change over the course of the study for each measured variable was assessed by a paired students t-test.

Results

There were no difference between the active and placebo groups in any of the measured variables at entrance in to the study. There was a reduction in the total cholesterol (7.2%) and low density lipoprotein (9.6%) in the treated group of men over the three month treatment period. There was no significant change in either the triglyceride or high density lipoprotein in the treated group. No significant changes in any variable were seen in those subjects receiving placebo.

| Active group | Before treatment | After treatment | |
|---|---|---|---|
| (n = l4) | Mean (SD) | Mean (SD) | P value |
| Cholesterol | 7.493 (0.745) | 6.950 (0.728) | P = 0.0183 |
| Triglyceride | 2.124 (0.924) | 2.070 (0.256) | P = N.S. |
| HDL | 1.390 (0.256) | 1.295 (0.248) | P = N.S. |
| LDL | 5.231 (0.791) | 4.729 (0.673) | P = 0.0341 |
| Placebo group | Before treatment | After treatment | |
| (n = 16) | Mean (SD) | Mean (SD) | P value |
| Cholesterol | 7.544 (0.77) | 7.269 (0.91) | P = N.S. |
| Triglyceride | 2.106 (0.968) | 2.015 (0.951) | P = N.S. |
| HDL | 1.317 (0.366) | 1.254 (0.201) | P = N.S. |
| LDL | 5.212 (0.521) | 5.231 (0.760) | P = N.S. |

Conclusions

Treatment with the active composition significant reduces total cholesterol in men with mildly elevated levels. The majority of this reduction is seen in the fall of the low density lipoprotein.

During the study there were no clinically important adverse reactions due to the various treatments investigated. This is of major importance as significant adverse reactions can occur with all the major groups of drugs used to treat cardiovascular disease and may prevent their use in some patients.

1. A pharmaceutical composition for stabilising atherosclerotic plaques, preventing new plaques forming and resolving existing atherosclerotic plaques comprising lycopene or a related carotenoid other than β-carotene, at least one flavonoid, an amino acid or derivative or precursor thereof, magnesium, ascorbate and tocopherol (vitamin E) or a derivative, precursor or isomer thereof.

2. The composition according to claim 1, wherein the lycopene is L-lycopene.

3. The composition according to claim 1, wherein the related carotenoid is selected from the group consisting of lutein and zeaxanthin.

4. The composition according to claim 1, wherein the flavonoid is selected from the group consisting of rutin, hesperedin, iproflavone, bioflavonoid mixture, flavones, flavonals, isoflavones or a mixture thereof.

5. The composition according to claim 1, wherein the amino acid is selected from the group consisting of lysine, arginine, proline or methionine.

6. The composition according to claim 5, wherein the amino acid is L-lysine.

7. The composition according to claim 1, wherein the magnesium is present as a magnesium salt.

8. The composition according to claim 1, wherein the ascorbate is selected from the group consisting of ascorbic acid, ascorbic acid ester, and a salt of ascorbic acid.

9. The composition according to claim 8, wherein the ascorbic acid salt is magnesium ascorbate.

10. The composition according to claim 1, wherein the derivatives, precursors or isomers of tocopherol are selected from the group consisting of acetate, tocophenols, d,l α-tocopherol and d-αtocopherol.

11. The composition according to claim 1, comprising in addition at least one constituent selected from the group consisting of garlic powder, coenzyme Q10 and other nutrients such as lipoic acid.

12. The composition according to claim 1, in unit dosage form.

13. A pharmaceutical composition for stabilising atherosclerotic plaques, preventing new plaques forming and resolving existing atherosclerotic plaques comprising L-lysine or salt, ester or complex thereof, L-magnesium ascorbate or L-ascorbic acid or a salt or ester thereof, α-tocopherol or an isomer or derivative thereof, lycopene or related carotenoid other than β-carotene, coenzyme Q10, bioflavonoid or complex, together with a pharmaceutically acceptable carrier therefor.

14. The composition according to claim 13, in unit dosage form.

15. A pharmaceutical composition for stabilising atherosclerotic plaques, preventing new plaques forming and resolving existing atherosclerotic plaques comprising from 20 to 60% by weight of amino acid or salt or ester thereof, from 20 to 60% by weight ascorbic acid or salt or ester thereof or 20 to 60% by weight magnesium ascorbate, from 5 to 20% by weight of tocopherol or isomer or ester thereof, from 0.2 to 1% by weight of lycopene or ester thereof or a related carotenoid other than β-carotene and from 4 to 12% by weight of flavonoid or complex thereof, and if magnesium ascorbate is not present, 2 to 6% by weight of a magnesium compound.

16. The pharmaceutical composition according to claim 15, in unit dosage form.

17. A pharmaceutical composition for stabilising atherosclerotic plaques, preventing new plaques forming and resolving existing atherosclerotic plaques in unit dosage form, said composition comprising from 150 to 450 mg d,l-α tocopheryl acetate, from 1.5 to 8.0 mg lycopene, from 2000 to 4000 mg L-lysine, from 1600 to 5000 mg magnesium ascorbate and from 300 to 900 mg bioflavonoid mixture.

18. The composition according to claim 17, wherein said composition is in the form of a sachet.

19. The composition according to claim 17, wherein said composition is incorporated into a nutritional product.

20. Use of the pharmaceutical composition according to any one of claims 1, 13, 15 or 17 for the manufacture of a medicament for the treatment of thromboembolic disease.

21. A method of treating thromboembolic disease comprising administering to a patient in need thereof a pharmaceutical formulation comprising lycopene or a related carotenoid other than β-carotene, at least one flavonoid, an amino acid or derivative or precursor thereof, magnesium, ascorbate and tocopherol or a derivative, precursor or isomer thereof.

22. The method of claim 21, wherein said formulation comprises from 20 to 60% by weight of amino acid or salt or ester thereof, from 20 to 60% by weight ascorbic acid or salt or ester thereof or 20 to 60% by weight magnesium ascorbate, from 5 to 20% by weight tocopherol or isomer or ester thereof, from 0.2 to 1% by weight of lycopene or ester thereof or a related carotenoid other than β-carotene and from 4 to 12% by weight of flavonoid or complex thereof and if magnesium ascorbate is not present 2 to 6% by weight of a magnesium compound.

23. The method according to claim 21, wherein the daily dose of the formulation comprises from 3 to 16 mg L-lycopene, from 3000 to 9000 mg L-ascorbic acid, from 300 to 900 mg magnesium, from 3000 to 9000 mg L-lysine as the monohydro-chloride, from 300 to 900 mg d,l-α-tocopheryl acetate and from 600 to 1800 mg of at least one flavonoid or a mixture thereof.

24. The method according to claim 23, wherein the daily dose is administered as divided doses 2, 3 or 4 times daily.

25. The method according to claim 21, wherein treating the thromboembolic disease comprises stabilising atherosclerotic plaques, reducing the size of said plaques and preventing the formation of new plaques.

26. The method according to claim 21, wherein said thromboembolic disease is angina, coronary insufficiency, myocardial infarction, arterial hypertension, arterial aneurysms, intermittent claudication, cerebral insufficiency, stroke or retinal thrombosis.

27. The method according to claim 21, wherein said formulation is co-administered with an agent for lowering blood lipids, a drug acting on platelet function or a non-steroidal anti-inflammatory agent.
